(19) United States
(12) Patent Application Publication
(10) Pub. No.: US 2006/0057688 A1
(43) Pub. Date: Mar. 16, 2006

(54) Method for separating vitamin K2 from Bacillus bacterium culture

(75) Inventors: Akiko Shimatani, Kumamoto (JP); Toshiharu Iwata, Kumamoto (JP); Mototake Tsutsui, Kumamoto (JP)

Correspondence Address: HOGAN & HARTSON LLP;IP GROUP, COLUMBIA SQUARE, 555 THIRTEENTH STREET, N.W., WASHINGTON, DC, 20004, US

(73) Assignee: CHISSO Corporation

(21) Appl. No.: 11/212,920

(22) Filed: Aug. 29, 2005

(30) Foreign Application Priority Data
Aug. 30, 2004 (JP) 2004-249825

(51) Int. Cl. C12P 7/66

(52) U.S. Cl. 435/133

(57) ABSTRACT

Vitamin K2 is separated from a cultured product of a Bacillus bacterium such as Bacillus natto by insolubilizing vitamin K2 contained in the cultured product using a metal ion of bivalent or more and separating the insolubilized vitamin K2 from the cultured product.

### BACKGROUND OF THE INVENTION

1. Field of the Invention

The present invention relates to a method for separating vitamin K2 from a cultured product of a bacterium belonging to the genus Bacillus such as Bacillus natto. The present invention also relates to a method for producing concentrated vitamin K2 and a method for producing culture extracts of a Bacillus bacterium with low content of vitamin K2.

2. Description of the Related Art

Natto, a traditional fermented food that has long been eaten in Japan, has a high nutritional value, and thus it is recently re-evaluated as a health food. One of the components which is in the spotlight is natto kinase, which was found as a thrombolytic enzyme in natto by Sumi et al. in 1987 (JP 2001-246546 A). Natto kinase itself acts as a fibrinolytic enzyme and lyses thrombus, and therefore, natto kinase is expected as an effective preventive medicine for cerebral thrombosis, cerebral infraction and the like, as well as for a long-flight syndrome. Natto kinase is an enzyme secreted from bacterial cells and can be obtained by culturing a Bacillus natto in a liquid medium In addition, natto contains natto kinase and culture extracts of Bacillus natto obtained by concentrating the culture of Bacillus natto are commercially available as a health food in the form of capsule, tablet, or the like.

Meanwhile, it has been known that natto contains vitamin K2 (also called as menaquinone). Vitamin K2 is supposed to be used for production of chemical energy, transport of electrolytes, intracellular electron transfer system, and the like in bacterial cells, and is usually not secreted from the bacterial cells. However, it is known that vitamin K2 is obtained in a form of an aqueous micelle binding to a membrane lipid in a fermented solution of Bacillus natto because Bacillus natto is easily subject to bacteriolysis. Although toxicity due to excessive ingestion of vitamin K2 has not been reported, vitamin K2 is pharmaceutically contraindicated in a subject administered with warfarin that is a therapeutic agent for thrombosis, because vitamin K2 is antagonistic to warfarin. For this reason, culture extracts of Bacillus natto with low content of vitamin K2 has been desired.

On the other hand, usefulness of vitamin K2 itself has also been receiving attention For example, recently, it was found that vitamin K2 activates osteocalcin, a protein present in bone, and promotes bone formation, therefore vitamin K2 is receiving attention as a preventive medicine for osteoporosis. However, a dosage of vitamin K2 required for preventing osteoporosis is as large as 45 mg per day, and the value fills under more than 10 kg of natto per day. Therefore, it is substantially impossible to take the required dosage of vitamin K2 for preventing osteoporosis from natto. Furthermore, there is also a problem that many consumers do not have a good feeling toward natto because of its distinctive odor, flavor, and texture. Consequently, it has been attempted to separate and concentrate vitamin K2 from natto, bacterial bodies of Bacillus natto, or a culture solution of Bacillus natto.

So far, as a method for reducing vitamin K2 content in culture extracts of Bacillus natto and a method for obtaining concentrated vitamin K2, some methods have been proposed: a method for extracting vitamin K2 from a cultured product of Bacillus natto using an organic solvent or a method for separating vitamin K2 by adsorbing vitamin K2 on activated carbon or silica gel and eluting vitamin K2 using an organic solvent (JP 2001-346546 A or JP 08-73396 A); a method for insolubilizing vitamin K2 using an organic solvent or salting-out (JP 11-92414 A); a method for separating vitamin K2 using chitosan (JP 2001-299277 A); a method for obtaining concentrated vitamin K2 by culturing a mutant strain having high vitamin K2-producing ability (JP 2000-83653 A). However, in such conventional methods, the stress on the environment is large because a large amount of organic solvents and salts are used, and procedures become complicated and production cost becomes high because the remaining organic solvents have to be completely removed. Furthermore, it was difficult to separate vitamin K2 and components other than vitamin K2 such as natto kinase, and collect them respectively from a cultured product Bacillus natto. For this reason, methods for separating vitamin K2 from a cultured product of Bacillus bacterium to obtain both vitamin K2 and the other components including natto kinase efficiently and with cheap cost and without using any organic solvent have been desired.

### SUMMARY OF THE INVENTION

An object of the present invention is to obtain a concentrated vitamin K2 and culture extracts of a Bacillus bacterium with low content of vitamin K2 by efficiently separating vitamin K2 from a cultured product of a Bacillus bacterium.

The inventors of the present invention have made extensive studies to develop a method for separating vitamin K2 easily, with less stress on the environment, and at cheaper cost compared to the conventional methods. Consequently, the inventors of the preset invention found that vitamin K2 can be efficiently separated from a cultured product of Bacillus bacterium by insolubilizing vitamin K2 contained in a cultured product of Bacillus natto using a metal ion of bivalent or more and collecting the insolubilized vitamin K2. In addition, the inventors of the present invention also found that the insolubilized vitamin K2 can be resolubilized by adding an anion which is capable of forming an insoluble salt with the metal ion and releasing vitamin K2, and thus completed the invention.

That is, the present invention is as follows.

(1) A method for separating vitamin K2 from Bacillus bacterium culture, comprising the steps of insolubilizing vitamin K2 contained in the culture using a metal ion of bivalent or more and separating the insolubilized vitamin K2 complex from culture broth.

(2) The method according to (1), wherein said Bacillus bacterium is Bacillus natto.

(3) The method according to (1) or (2), wherein said metal ion is at least one kind of metal ion selected from the group consisting of calcium ion, magnesium ion, zinc ion, manganese ion, iron ion, and aluminum ion.

(4) The method according to (1) or (2), wherein said metal ion is calcium ion or magnesium ion.

(5) The method according to one of (1) to (4), wherein vitamin K2 is insolubilized by adding 0.07 to 3% by weight of the metal ion with respect to the culture.

(6) The method according to one of (1) to (4), wherein vitamin K2 is insolubilized by adding 0.14 to 1.4% by weight of the metal ion with repect to the culture.

(7) The method according to one of (1) to (4), wherein vitamin K2 is insolubilized by adding 0.14 to 0.28% by weight of the metal ion with respect to the culture.

(8) The method according to one of (1) to (7), wherein vitamin K2 is insolubilized under conditions of pH 7 to 9.

(9) A method of producing Bacillus bacterium culture extract with low content of vitamin K2, comprising the steps of removing vitamin K2 complex from Bacillus bacterium culture by separating the vitamin K2 complex using the method according to one of (1) to (8), and collecting the resultant Bacillus bacterium culture extracts.

(10) Bacillus bacterium culture exacts with low content of vitamin K2, which is obtainable by removing vitamin K2 complex from Bacillus bacterium culture by separating the vitamin K2 complex using the method according to one of (1) to (8).

(11) A method for producing vitamin K2, comprising the steps of insolubilizing vitamin K2 contained in a cultured product of a Bacillus bacterium culture using he method according to one of (1) to (8), resolubilizing the insolubilized vitamin K, and collecting vitamin K2.

(12) A method for producing concentrated vitamin K2, comprising the steps of insolubilizing vitamin K2 contained in a Bacillus bacterium culture using the method according to one of (1) to (8), resolubilizing the insolubilized vitamin K2 in a solution, and demineralizing and concentrating the vitamin K2 solution.

(13) The method according to (11), wherein vitamin K is resolubilized by using an anion that is capable of forming an insoluble salt together with the metal ion that forms the insolubilized vitamin K2 complex and is capable of releasing vitamin K2 from the insolubilized vitamin K2 complex.

(14) The method of (13), wherein said anion is carbonate ion or phosphate ion.

By using a separating method of the present invention, both culture extracts of Bacillus bacterium with low content of vitamin K2 and concentrated vitamin K2 can be obtained efficiently from a cultured product of Bacillus bacterium. According to the present invention, culture extracts of Bacillus bacterium with low content of vitamin K and concentrated vitamin K2 can be more efficiently obtained compared to the conventional methods using an organic solvent.

### DETAILED DESCRIPTION OF THE INVENTION

Hereinafter, the present invention will be described in detail.

The separation method of the present invention is a method for separating vitamin K2 from a cultured product of Bacillus bacterium (Bacillus bacterium culture), comprising the steps of insolubilizing vitamin K2 contained in the cultured product using a metal ion of bivalent or more and separating the insolubilized vitamin K2 (insolubilized vitamin K2 complex) From the cultured product.

A bacterium used in the present invention may be any species as far as it belongs to the genus Bacillus that produces vitamin K2. A bacterium used for production of foods such as Bacillus natto (also called as Bacillus subtilis natto) is particularly preferable when it is provided for food. A commercially available Bacillus bacterium may be used. Alternatively, a bacterium available from public microorganism depositary institutes may also be used. For example, Bacillus subtilis NBRC16449 strain, Bacillus subtilis NBRC 3335 strain, or the like obtainable from Biological Resource Center of National Institute of Technology and Evaluation (2-5-8 Kazusakamatari, Kisarazu-shi, Chiba, 292-0818 JAPAN) may be used.

The cultured product may be solid or liquid, and a cultured solution of Bacillus bacterium may be preferably used in the present invention. Culture media and culture methods for culturing Bacillus bacterium are not particularly limited. Soybean powder, yeast extract, peptone and the like may be used as a nitrogen source, but a low molecule of nitrogen source such as peptone, oligopeptide, and polypeptide is preferable from the viewpoint of handiness such as solubility to water. A culture medium may be a medium generally used in fermentation industry such as those containing sucrose, starch, glucose, or glycerin as a carbon source; other minerals and vitamins. Components of a culture medium are preferably foods or food additives because vitamin K2 and culture extracts may be provided as food components.

In a case where a cultured product is solid such as natto, it is preferable to suspend the solid cultured product such as natto in an aqueous solution by adding water and grinding as a pretreatment. For example, an active ingredient in the fermented product such as natto kinase, and vitamin K2 are efficiently extracted into an aqueous solution by the pretreatment of adding water, grinding and mixing well.

By adding a metal ion of bivalent or more to such a culture solution containing vitamin K2 as described above or an aqueous solution containing vitamin K2, vitamin K2 is insolubilized. Such a culture solution or aqueous solution may be a solution from which solid components have been removed or a solution containing solid components such as cells of the Bacillus bacterium. pH of the solution during the insolubilization reaction is not particularly limited, but pH of 3 to 11 is preferable, pH of 6 to 10 is more preferable, and pH of 7 to 9 is particularly preferable. The insolubilization of vitamin K2 can be performed by adding a metal ion of bivalent or more into a cultured solution of Bacillus bacterium, and can also be performed by contacting a column (carrier) carrying the metal ion with the solution.

Examples of the metal ion of bivalent or more include calcium ion, magnesium ion, zinc ion, manganese ion, iron ion and aluminum ion, which can be used singly or in combination. Among these, calcium ion and magnesium ion are preferable because they are safely contained in food composition. Calcium ion or magnesium ion may be used in a form of a metallic salt (including a hydrate of metallic salt) such as calcium chloride or magnesium chloride. The metal ion is preferably added in an amount of 0.07 to 3% by weight, more preferably 0.14 to 1.4% by weight, particularly preferably 0.14 to 028% by weight with respect to the cultured product.

Furthermore, anionic aggregation-promoting agents such as alginic acid can be used in combination with the metal ion for insolubilization, because anionic aggregation-promoting agents such as alginic acid enhance aggregation and help to remove insoluble products. In this case, the above-mentioned metal ion (metallic salt) is preferably added after alginic acid is added and dissolved.

Vitamin K2 can be separated from a cultured product of Bacillus bacterium by collecting the generated insolubilized product from the solution. Examples of {he method of collecting the insolubilized vitamin K2 (metallic salt of vitamin K2) from the solution include centrifugation, membrane separation, and decantation. The insolubilized vitamin K2 collected by separation may be washed with an aqueous solution such as water or physiological saline solution so long as the insolubilized vitamin K2 is not released again.

Vitamin K2 can be collected as complex by resolubilizing the insolubilized vitamin K2. The concentrated vitamin K2 can be obtained by resolubilizing the insolubilized vitamin K2, and demineralizing and concentrating the obtained vitamin K2 solution.

For example, vitamin K2 can be collected as the complex as described below. First, the above-described insolubilized vitamin K2 is resuspended in an aqueous solution such as water or a physiological saline solution Then, the insolubilized vitamin K2 is resolubilized by adding an anion to the suspension. The anion is not particularly limited so long as it can solubilize and release vitamin K2 in the insolubilized vitamin K2 complex. In particular, an anion that is capable of forming an insoluble salt with the above-mentioned metal ion that forms the insolubilized vitamin K2 complex is preferable for efficient separation of vitamin K2. Examples of such an anion include a carbonate ion and phosphate ion, which can be used alone or in combination. The anion can be used in a form of a salt such as sodium carbonate or potassium carbonate. The anion may be added in such an amount that the insolubilized vitamin K2 is resolubilized. For example, 0.2 to 5% by weight of carbonate ion with respect to the above-mentioned suspension may be added. Alternatively, the resolubilization can be performed by directly suspending the insolubilized vitamin K2 in an aqueous solution containing such an anion.

Next, the resolubilized vitamin K2 is collected. This step can be performed by removing an insoluble salt generated by addition of the anion. Examples of a method of removing insoluble materials containing such an insoluble salt include centrifugation and membrane separation The obtained supernatant or filtrated solution containing vitamin K2 is preferably demineralized by typical methods such as ultrafiltration, gel filtration, dialysis, and ion exchange chromatography, to thereby remove salts existing in the solution.

The obtained vitamin D-containing solution may be concentrated by such methods as reverse osmosis membrane concentration and vacuum concentration, to thereby obtain a concentrated vitamin K2. By the method of the present invention, vitamin K2 may be collected at a recovery rate of 40% or more, and a vitamin K2 concentrate containing 500 to 2,000 mg/kg of vitamin K2 may be obtained from a cultured solution of the Bacillus bacterium.

The concentrated vitamin K2 obtained by the method of the present invention is derived from a raw material of natto, a traditional fermented food. Therefore, it can be used as an ingredient of drink, foods, and in particular, health foods, and also used for preventing a disease related to vitamin K2 such as osteoporosis.

By the way, the separation of the insolubilized vitamin K2 from the cultured product or Bacillus bacterium leads to removal of 95% or more, or in some cases, 99% or more of vitamin K2 ftom the cultured product of Bacillus bacteterium. That is, culture ertracts of Bacillus bacterium with low content of vitamin K2 can be obtained by removing the insolubilized vitamin K2 from the cultured product, and preferably by further removing salts remained in the resultant supernatant or filtrated solution by demineralizing the supernatant or the filtrated solution with a general method such as ultrafiltration, gel filtration, or dialysis. Finally, by concentrating the obtained solution using a method such as reverse osmosis membrane concentration or vacuum concentration, culture extracts of Bacillus bacterium with low content of vitamin K2 can be obtained. The culture acts of Bacillus bacterium may be a liquid, paste, or powder. In the present invention, the “culture exacts of Bacillus bacterium with low content of vitamin K2” refers to culture extracts of Bacillus bacterium which has a lower content of vitamin K2 than typical culture extracts of Bacillus bacterium produced by conventional extraction methods. Examples of the “culture extracts of Bacillus bacterium with low content of vitamin K2” include culture extracts of Bacillus bacterium having a vitamin K2 content of 1 mg/kg or less, preferably a vitamin K2 content of 0.2 mg/kg or less.

Culture extracts of Bacillus natto obtained by the present invention is derived from natto, a raw material of a traditional fermented food, therefore it can be used as an ingredient of drink, foods and, in particular, health foods.

### EXAMPLES

Hereinafter, the present invention will be described in detail by referring to the examples. However the scope of the present invention is not limited to the examples.

Method for Determining Vitamin K2 Content

0.8 mL of methanol was added to 0.2 mL of a test sample and strongly stir The mixture was subjected to centrifugation at 6,000×g for 10 minutes to collect supernatant, and the content of vitamin K2 in the supernatant was determined. In a case of a dry sample, a sample of predetermined weight was placed in a mortar, added with ethanol, and ground well with a pestle. Subsequently, filtration was carried out with a filter paper (manufactured by Advantec Toyo Kaisha, Ltd., quantitative filter paper No. 5A) and the filtrated solution was concentrated under pressure, followed by adjusting a volume with methanol and determining the concentration of vitamin K2 contained in the solution. The determination was performed by a high performance liquid chromatography (manufactured by Agilent Technologies, Ltd., HP1100) under the following conditions; column: Wakosil-II5C18 AR (manufactured by Wako Pure Chemical Industries, Ltd.), mobile phase: methanol, temperature: 40° C., flow rate: 1 mL/min., and detection: 248 nm. The concentration of vitamin K2 was determined on the basis of a calibration curve calculated using a menaquinone-7 standard (content of 99.5%, manufactured by Honen Corporation).

### Example 1

Separation of Vitamin K2 from a Cultured Product of Bacillus natto Using Various Metallic Salts

A commercially available strain of Bacillus natto (Miyagino strain, produced by Miyagino Natto Seizosho) was precultured by shaking in a 500 mL Sakaguchi flask containing a culture solution (pH 7.2) consisting of 2% of soybean peptone, 0.3% of yeast extract, and 3% of glycerol at 37° C. for 24 hours. Subsequently, the precultured solution whose amount was 1% of culture solution was inoculated into culture solution (pH 7.2) consisting of 1% of soybean peptone, 0.5% of yeaat extract and 6% of glycerol, and subjected to a submerged culture in a 50 L jar at 37° C. for 20 hours. After the culture, the culture solution was subjected to separation using a microfiltration membrane having pore sizes of 1 gm to scparatc bacterial cells, and thereby, the filtred solution with vitamin K2 concentration of 4.5 mg/L was obtained. The filtrated solution was adjusted to pH 8.0, and divided into 6 flasks each containing 5 mL. 1% by weight of each of potassium chloride, sodium chloride, ammonium sulfate, manganese sulfate heptahydrate, zinc sulfate heptahydrate, and calcium chloride dihydrate was added to each flask and the flasks were stirred for 1 hour. Subsequently, the samples were subjected to centrifugation at 6,000×g for 10 minutes to separate precipitates containing insolubilized vitamin K2 from the supernatant Vitamin K2 concentration in each supernatant (supernatant 1) was analyzed. Meanwhile, 1% solution of sodium carbonate was added to each precipitate to resolubilize vitamin K2, and the obtained solution was stirred over 1 hour, followed by centrifugation to obtain a supernatant. Vitamin K2 in the obtained supernatant (supernatant 2) was analyzed. The results are shown in Table 1. In Table 1, N.D. represents “Not Determined” because of no observation of precipitates.

TABLE 1
Content and Yield of Vitamin K2 after treated with
Various Metallic Salts and Sodium Carbonate

| Kind of salt | Sample | Vitamin K2 content | Yield |
|---|---|---|---|
| No addition | Microfiltrated | 4.5 mg/L | 100% |
| | solution of the | | |
| | culture solution | | |
| Potassium salt | Supernatant 1 | 4.2 mg/L | 94% |
| (+sodium carbonate) | Supernatant 2 | N.D. | N.D. |
| Sodium salt | Supernatant 1 | 4.2 mg/L | 95% |
| (+sodium carbonate) | Supernatant 2 | N.D. | N.D. |
| Ammonium salt | Supernatant 1 | 4.2 mg/L | 94% |
| (+sodium carbonate) | Supernatant 2 | N.D. | N.D. |
| Manganese salt | Supernatant 1 | 0.05 mg/L or less | 5% or less |
| (+sodium carbonate) | Supernatant 2 | 2.0 mg/L | 44% |
| Zinc salt | Supernatant 1 | 0.05 mg/L or less | 5% or less |
| (+sodium carbonate) | Supernatant 2 | 1.9 mg/L | 43% |
| Calcium salt | Supernatant 1 | 0.05 mg/L or less | 5% or less |
| (+sodium carbonate) | Supernatant 2 | 1.7 mg/L | 37% |

As shown in Table 1, almost no virgin K2 was did in supernatant 1 with addition of the salts of bivalent metallic ions such as manganese ion, zinc ion and calcium ion, because vitamin K2 became insoluble by the ions and was precipitated by centrifugation. In addition, vitamin K2 was resolubilized and recovered in the supernatant 2 by adding sodium carbonate to the precipitates.

On the other hand, the addition of ammonium sulfite, which is commonly used for salting out, or potassium chloride or sodium chloride, which is a salt of monovalent metallic ion, did not precipitate vitamin K2, and therefore vitamin K remained in the supernatant 1 and could not be separated.

### Example 2

Separation of Vitamin K2 from a Cultured Product of Bacillus natto Using a Calcium Salt

A commercially available Bacillus natto (Miyagino strain, produced by Miyagino Natto Seizosho) was precultured by shaking in a 500 mL Sakaguchi flask containing a culture solution (pH 72) consisting of 2% of soybean peptone, 0.3% of yeast extract and 3% of glycerol at 37° C. for,24 hours. Subsequently, the precultured solution whose amount was 1% of culture solution was inoculated into a culture solution 6H 7.2) consisting of 1% of soybean peptone, 0.5% of yeast extract, and 6% of glycerol, and subjected to a submerged culture in a 50 L jar at 37° C. for 20 hours. After the culture, 5 g of sodium alginate (100 to 150 cp) was added to the 20 L of the culture solution and stitred for 1 hour. Then, 500 g of calcium chloride dihydrate was added to the solution and sred for 1 hour. Then, the solution was subjected to separation using a microfiltration membrane having pore sizes of 1 μm, and thereby, a filtrated solution with the vitamin K2 concentration of 0.075 mg/L or less was obtained. The filtrated solution was concentrated and dialyzed against pure water using a dialysis membrane having molecular cutoff of 6,000 to 8,000 (Spectra/Pro® Membrane, manufactured by Spectrum Labs, Inc.) for 4 hours to demineralize the filtrated solution. Then, 0.5% by weight of an activated carbon is added and stirred for decolorization, followed by freeze-drying. Consequently, 5.7 g/L of pale yellow powder having a slight odor with a vitamin K2 content of 0.05 mg/kg or less was obtained.

Accordingly, by adding sodium alginate and calcium chloride to a cultured product of Bacillus natto, vitamin K2 became insoluble and was removed. As a result, culture extracts of Bacillus natto with low content of vitamin K2 was produced from a cultured solution of Bacillus natto.

### Comparative Example

The cultured solution or Bacillus natto was obtained in the same procedure of Example 2. After the culture, the culture solution was directly subjected to separation using a microfiltration membrane having pore sizes of 1 μm without adding sodium alginate and calcium chloride. As a result, the concentration of vitamin K2 in the filtrated solution was 4.6 mg/L. Then, the filtrated solution was concentrated and dialyzed, treated with an activated carbon, and freeze-dried in the same manner as Example 2, and as a result, 5.6 g/L (5.6 g per 1 L of he culture solution) of pale yellow powder having a slight odor with a vitamin K2 content of 1.9 mg/kg was obtained.

### Example 3

Production of Concentrated Vitamin K2

A commercially available Bacillus natto ( hi strain, produced by Takahashi Yuzo Labo) was precultured by shaking in a 500 mL Sakaguchi flask containing a culture solution (pH 72) consisting of 2% of soybean peptone, 0.3% of yeast extrc and 3% of glycerol at 37° C. for24 hours. Subsequently, the precultured solution whose amount was 1% of culture solution was inoculated into culture solution <pH 7.2) consisting of 1% of soybean peptone, 0.5% of yeast extract, and 4% of glycerol, and subjected to a submerged culture in a 50 L jar at 37° C. for 20 hours. After the culture, the culture solution was subjected to ce trifugation at 6,000×g for 10 minutes, and thereby a supernatant with a vitamin K2 concentration of 4.0 mg/L was obtained. Then, 0.9 g of calcium chloride dihydrate was added to 180 mL of the supernatant and stirred for 1 hour, then centrifuged at 6,000×g for 10 minutes, to thereby separate the precipitate containing insolubilized vitamin K2 from a supernatant. The precipitate was resuspended to pure water and 0.9 g of sodium carbonate was added to resolubilize vitamin K2. Then, the suspension was stirred for 1 hour, followed by centrifugation at 6,000×g for 10 minutes. The resultant supernatant was subjected to dialysis against pure water for 4 hours using a dialysis membrane having molecular cutoff of 6,000 to 8,000 (Spectra/Pro® Membrane, manufactured by Spectrum Labs, Inc.). By freeze-drying the dialyzed product, 150 mg of powder containing 1,800 mg/kg of vitamin K2 was obtained. Accordingly, concentrated vitamin K2 was obtained.

### Example 4

Production of Culture Extracts of Bacillus natto with Low Content of Vitamin K2 and Concentrated Vitamin K2

Cells of Bacillus subtilis NBRC16449 stain was scraped from a slant with a platinum loop and inoculated into a 500 mL Sakaguchi flask containing a culture solution (pH 7.2) consisting of 1% of soybean peptone, 0.5% of yeast extra and 4% of glycerol and cultured at 37° C. for 32 hours. After the culture, the culture solution was subjected to centrifugation at 6,000×g for 10 minutes, and thereby a superntant with a vitamin K2 concentration of 2.1 mg/L was obtained. 80 mL of the obtained supernatant was adjusted to pH 9.0, and added with 0.8 g of calcium chloride dehydrate, and stirred for 1 hour. The solution was subjected to centrifugation at 6,000×g for 10 minutes, to separate precipitate and a supernatant. The supernatant was filtrated with a filter made of cellulose-ace having pore sizes of 0.45 μm (manufactured by Advantec Toyo Kaisha, Ltd.), followed by concentration and dialysis against pure water for 6 hours with a dialysis membrane having molecular cutoff of. 6,000 to 8,000 (Spectra/Pro® Membrane, manufactured by Spectrum Labs, Inc.). By freeze-drying the dialyzed product, 0.2 g of powder with vitamin K2 content of 1 mg/kg or less was obtained. Accordingly, Bacillus natto culture extracts with low content of vitamin K2 was obtained.

Meanwhile, the precipitate obtained by the above centrifugation step was resuspended in pure water. 1.6 g of sodium carbonate was added thereto and stirred for 1 hour, followed by centrifugation at 6,000×g for 10 minutes. The resultant supernatant was dialyzed against pure water for 6 hours with a dialysis membrane having molecular cutoff of 6,000 to 8,000 (Spectrum/Pro Membrane, manufactured by Spectrum Labs, Inc.). By freeze-drying the dialyzed product, 60 mg of powder with vitamin K2 content of 1,600 mg/kg was obtained. Accordingly, a concentrated vitamin K2 was obtained.

While the invention has been described in detail with reference to preferred embodiments thereof, it will be apparent to one skilled in the art that various changes can be made, and equivalents employed, without departing from the scope of the invention. All the references cited herein, including the priority document, JP 2004249825, are incorporated as a part of this application by reference.

What is claimed is:

1. A method for separating vitamin K2 from Bacillus bacterium culture, comprising the steps of insolubilizing vitamin K2 contained in the culture using a metal ion of bivalent or more, and separating the insolubilized vitamin K2 complex from culture broth.

2. The method according to claim 1, wherein said Bacillus bacterium is Bacillus natto.

3. The method according to claim 1, wherein said metal ion is at least one kind of metal ion selected from the group consisting of calcium ion, magnesium ion, zinc ion, manganese ion, iron ion, and aluminum ion.

4. The method according to claim 1, wherein said metal ion is calcium ion or magnesium ion.

5. The method according to claim 1, wherein vitamin K2 is insolubilized by adding 0.07 to 3% by weight of the metal ion with respect to the culture.

6. The method according to claim 1, wherein vitamin K2 is insolubilized by adding 0.14 to 1.4% by weight ofthe metal ion with respect to the culture.

7. The method according to claim 1, wherein vitamin K2 is insolubilized by adding 0.14 to 0.28% by weight of the metal ion with respect to. the culture.

8. The method according to claim 1, wherein vitamin K2 is insolubilized under conditions of pH 7 to 9.

9. A method for producing Bacillus bacterium culture extra with low content of vitamin K2, comprising the steps of removing vitamin K2 complex from Bacillus bacterium culture by separating the vitamin K2 complex using the method according to claim 1 and collecting the resultant Bacillus bacterium culture extracts.

10. Bacillus bacterium culture extracts with low content of vitamin. K2, which is obtainable by removing vitamin K2 complex from Bacillus bacterium culture by separating the vitamin K2 complex using the method according to claim 1.

11. A method for producing vitamin K2, comprising the steps of insolubilizing vitamin K2 contained in Bacillus bacterium culture using the method according to claim 1, resolubilizing the insolubilized vitamin K2, and collecting vitamin K2.

12. A method for producing concentrated vitamin K2, comprising the steps of insolubilizing vitamin K2 contained in Bacillus bacterium culture using the method according to claim 1, resolubilizing the insolubilized vitamin K2 in a solution, and demineralizing and concentrating the vitamin K2 solution.

13. The method according to claim 11, wherein vitamin K2 is resolubilized by using an anion that is capable of forming an insoluble salt together with the metal ion that forms the insolubilized vitamin K2 complex and is capable of releasing vitamin K2 from he insolubilized vitamin K2 complex.

14. The method of claim 13, wherein said anion is carbonate ion or phosphate ion
